(19) United States
(12) Patent Application Publication
(10) Pub. No.: US 2021/0269626 A1
(43) Pub. Date: Sep. 2, 2021

(54) BREATHABLE ELASTIC FILM AND LAMINATES AND ARTICLES PREPARED THEREFROM

(71) Applicant: DOW GLOBAL TECHNOLOGIES LLC, Midland, MI (US)

(72) Inventors: Glenn POLLEFEYT, Terneuzen (NL); Barbara BONAVOGLIA, Horgen (CH); Eduardo ALVAREZ, Tarragona (ES)

(21) Appl. No.: 17/258,272

(22) PCT Filed: Jul. 19, 2019
(86) PCT No.: PCT/US2019/042517

§ 371 Date: Jan. 6, 2021

(30) Foreign Application Priority Data
Jul. 19, 2018 (EP) 18382543.9

(51) Int. Cl. C08L 23/08; C08K 3/26

(52) U.S. Cl. CPC C08L 23/0815; C08L 71/02; C08L 2203/16; C08K 3/26

(57) ABSTRACT

A breathable elastic film includes particular amounts of a polyolefin elastomer having a density of 0.860 to 0.890 grams per cubic centimeter, a polyethylene oxide having a Brookfield viscosity in 5 weight percent aqueous solution at 25° C. of 30 to 115 centiPoise, and a hydrophilic filler. The film has a water vapor transmission rate of at least 1,000 g-mil/m²-day, as determined according to ASTM E398-2780. Composite laminates and articles including the films are also disclosed.

### BACKGROUND

Water vapor permeable polyolefin films have utility in various applications. For example, films that provide a liquid barrier but high water vapor transmission are widely used in, for example, hygiene, medical, protective garment, and building and construction markets. Disposable hygiene and medical applications such as baby diapers, adult incontinence products, and breathable barrier surgical gowns require cost effective solutions to achieve high water vapor transport rate (WVTR). Typical levels of breathability are reported to range from 500 g/m²-day to 20,000 g/m²-day depending upon the application and test methods.

For existing breathable films, the addition of fillers like CaCO₃ into polyethylene has been used to make moisture breathable films of high WVTR, but this requires a post-orientation process, such as machine direction orientation or the use of inter-digitating or intermeshing rollers, also called “ring rolling”, to create cavitation around the filler particles (see for example, WO2007/081548 or WO1998/004397). These films therefore need to be intrinsically non-elastic and they should not recover to their original shape upon stretching.

Existing elastic films include low crystallinity polyolefin plastomers, which typically have WVTR values of only 30 to 50 g/m²-day. Such films tend to be tacky and elastic. Their typical use in hygiene absorbent products is a closure systems, or elastic ears, in open diapers or as elastic side panels in pull-up diapers. These films are considered non-breathable, but give an excellent elastic recovery upon stretching.

High levels of WVTR may also be achieved using polymers having intrinsically higher level of permeability to moisture. These polymers typically have hydrophilic functional groups exhibit high permeation to water vapor. For example polyamide (nylon) films, are already used in fruit packaging applications. These films do provide high WVTR values (about 300 g/m²-day) combined with toughness and good optics, but compared to polyolefins they are more expensive and are more difficult to process. Accordingly, nylon films are not cost-effective in large-volume hygiene absorbent product applications.

Accordingly, there remains a need for films of that offer both breathability and elasticity, particularly for use in hygiene applications.

### BRIEF DESCRIPTION

A breathable elastic film comprises 30 to 45 weight percent of a polyolefin elastomer having a density of 0.860 to 0.890 grams per cubic centimeter; 10 to 15 weight percent of a polyethylene oxide having a Brookfield viscosity in 5 weight percent aqueous solution at 25° C. of 30 to 115 centiPoise; and 40 to 60 weight percent of a hydrophilic filler; wherein weight percent is based on the total weight percent of materials present in the film; and wherein the film has a water vapor transmission rate of at least 1,000 g-mil/m²-day, as determined according to ASTM E398-2780.

A composite laminate comprises the breathable elastic film.

A hygiene article comprises the breathable elastic film.

The above described and other features are exemplified by the following figures, detailed description, examples, and claims.

### BRIEF DESCRIPTION OF THE DRAWINGS

The following figures are exemplary embodiments.

FIG. 1 shows water vapor transmission rates of the films described herein.

FIG. 2 shows the elastic properties in the machine (MD) and cross (CD) directions of the films described herein.

### DETAILED DESCRIPTION

The present inventors have unexpectedly discovered a breathable elastic film is obtained when a polyolefin elastomer, a polyethylene oxide, and a hydrophilic filler are present in particular amounts. Specifically, the breathable elastic films comprise 30 to 45 weight percent of a polyolefin elastomer having a density of 0.860 to 0.890 grams per cubic centimeter, 10 to 15 weight percent of a polyethylene oxide having a Brookfield viscosity in 5 weight percent aqueous solution at 25° C. of 30 to 115 centiPoise, and 40 to 60 weight percent of a hydrophilic filler. The films may have an advantageous combination of elastic response and breathability.

The polyolefin elastomer can be present in an amount of 30 to 45 weight percent, based on the total weight percent of materials present in the film. Within this range, the polyolefin elastomer can be present in an amount of 32 to 43 weight percent.

In embodiments herein, the polyolefin elastomer is an ethylene-based elastomer, a propylene-based elastomer, or a combination thereof. The ethylene-or propylene-based elastomer may include a combination of ethylene and propylene, and may further include a comonomer, i.e., an additional polymerizable monomer other than ethylene or propylene. Examples of suitable comonomers include straight-chain or branched α-olefins of 3 to 30, preferably 3 to 20, carbon atoms, such as propylene, 1-butene, 1-pentene, 3-methyl-1-butene, 1-hexene, 4-methyl-1-pentene, 3-methyl-1-pentene, 1-octene, 1-decene, 1-dodecene, 1-tetradecene, 1-hexadecene, 1-octadecene and 1-eicosene; cycloolefins of 3 to 30, preferably 3 to 20, carbon atoms, such as cyclopentene, cycloheptene, norbornene, 5-methyl-2-norbornene, tetracyclododecene, and 2-methyl-1,4,5,8-dimethano-1,2,3,4,4a, 5,8,8a-octahydronaphthalene; di- and polyolefins, such as butadiene, isoprene, 4-methyl-1,3-pentadiene, 1,3-pentadiene, 1,4-pentadiene, 1,5-hexadiene, 1,4-hexadiene, 1,3-hexadiene, 1,3-octadiene, 1,4-octadiene, 1,5-octadiene, 1,6-octadiene, 1,7-octadiene, ethylidenenorbornene, vinyl norbornene, dicyclopentadiene, 7-methyl-1,6-octadiene, 4-ethylidene-8-methyl-1,7-nonadiene, and 5,9-dimethyl-1,4,8-decatriene; and 3-phenylpropene, 4-phenylpropene, 1,2-difluoroethylene, tetrafluoro ethyl ene, and 3,3,3-trifluoro-1-prop ene.

In some embodiments, polyolefin elastomer may be an olefin block copolymer (OBC) comprising two or more chemically distinct regions or segments (“blocks”) preferably joined in a linear manner, rather than in pendent or grafted fashion. OBCs may be produced via a chain shuttling process, and are described in U.S. Pat. Nos. 7,858,706, 7,608,668, 7,893,166, and 7,947,793. OBCs are characterized by unique distributions of both polydispersity (PDI, or Mw/Mn), block length distribution, and/or block number distribution, due, in an embodiment, to the effect of the shuttling agent in combination with multiple catalysts used in their preparation. In some embodiments, the OBC may be represented by the formula (AB), where n is at least 1, preferably an integer greater than 1, such as 2, 5, 10, 20, 50, 100, or higher, “A” a hard block and “B” is a soft block or segment.

The OBCs may include various amounts of hard and soft segments. “Hard” segments are blocks of polymerized units in which ethylene or propylene is present in an amount greater than 95 weight percent (wt %), or greater than 98 wt %, each based on the weight of the OBC, up to 100 wt %. The remainder may be comonomer, which may be absent in some embodiments. “Soft” segments are blocks of polymerized units including a comonomer in an amount of greater than 5 wt %, or greater than 10 wt %, or greater than 20 wt %, or greater than 40 wt %, or greater than 60 wt %, and may be up to 100 wt %, each based on the weight of the OBC. The soft segments may be present in the OBCs in an amount of 1 to 99 wt %, or 10 to 90 wt %, or 30 to 70 wt %, or 40 to 60 wt %, or 45 to 55 wt %, each based on the weight of the OBC. Conversely, the hard segments may be present in similar ranges. The weight percent of the soft segment and the hard segment may be calculated based on data obtained from differential scanning calorimetry (DSC) or nuclear magnetic resonance (NMR) spectroscopy. Such methods and calculations are disclosed in, for example, U.S. Pat. No. 7,608,668.

### Ethylene-Based Elastomer

In some embodiments the polyolefin elastomer is an ethylene-based elastomer in which ethylene comprises the majority mole fraction of the polyolefin elastomer, i.e., ethylene comprises at least 50 mole percent (mol %) of the whole polymer. More preferably ethylene comprises at least 60 mol %, at least 70 mol %, or at least 80 mol %, with the substantial remainder of the whole polymer comprising at least one other comonomer that is preferably an α-olefin having 3 or more carbon atoms, for example, propylene or octene. In some embodiments, the ethylene-based elastomer may comprise 50 to 90 mol % ethylene, preferably 60 to 85 mol %, or more preferably 65 to 80 mol %.

In an embodiment, the ethylene-based elastomer is an ethylene/α-olefin block copolymer comprising polymerized ethylene and one α-olefin as the only monomer types. In a further embodiment, the α-olefin is propylene, 1-butene, 1-hexene, or 1-octene, preferably propylene or 1-octene, more preferably 1-octene.

The ethylene/α-olefin block copolymer may have a melt index (MI or 12) from from 0.1 to 50 grams per 10 minutes (g/10 min), or from 0.3 to 30 g/10 min, or from 0.5 to 20 g/10 min, or from 0.5 to 10 g/10 min, each as measured according to ASTM D1238 at 190° C. using a load of 2.16 kg). In some embodiments the ethylene/alpha-olefin block copolymer may have a melt index from 0.5 to 10 g/10 min, as measured according to ASTM D1238 (230° C./2.16 kg). In some embodiments the ethylene/alpha-olefin block copolymer may have a melt index from 1.0 to 15 g/10 min, as measured according to ASTM D1238 (230° C./2.16 kg). The ethylene-based elastomer may have a density of 0.860 to 0.890 grams per cubic centimeter (g/cc), or 0.860 to 0.880 g/cc as measured according to ASTM D792.

Examples of suitable ethylene-based elastomers may include INFUSE™ 9007, INFUSE™ 9010, INFUSE™ 9107, INFUSE™ 9100, INFUSE™ 9507, INFUSE™ 9500, INFUSE™ 9807, ENGAGE™ 8100, ENGAGE™ 8200, ENGAGE™ 8150, AFFINITY™ EG 8100G, and AFFINITY™ EG 8200G, all of which are commercially available from The Dow Chemical Company (Midland, Mich.); may also include QUEO™ 6800 LA, QUEO™ 7001 LA, and QUEO™ 8203, all of which are commercially available from Borealis (Vienna, Austria); and may also include EXACT™ 4053 and EXACT™ 4049, all of which are commercially available from ExxonMobil Chemical Company (Spring, Tex.).

### Propylene-Based Elastomers

In some embodiments the polyolefin elastomer is a propylene-based elastomer in which propylene comprises the majority mole fraction of the polyolefin elastomer, i.e., propylene comprises at least 50 mol % of the whole polymer. More preferably propylene comprises at least 60 mol %, at least 70 mol %, or at least 80 mol %, with the substantial remainder of the whole polymer comprising ethylene or at least one other comonomer that is an α-olefin more than 3 carbon atoms, for example, 1-hexene or 1-octene. In some embodiments, the propylene-based elastomer may comprise 50 to 90 mol % propylene, preferably 60 to 85 mol % propylene, or more preferably 65 to 80 mol % propylene. When ethylene is present, the propylene-based elastomer may have from 3 to 15 mol % of ethylene, or from 5 to 14 mol % of ethylene, or 7 to 12 mol % ethylene. In some embodiments no comonomer is present in addition to the ethylene.

The propylene-based elastomer may have a melt flow rate (MF) from The ethylene/alpha-olefin block copolymer may have a melt index from 1.0 to 15 g/10 min, as measured according to ASTM D1238 at 230° C. using a load of 2.16 kg. The density of the propylene-based elastomer may be of 0.860 to 0.890 grams per cubic centimeter (g/cc), or 0.860 to 0.880 g/cc, as measured according to ASTM D792.

Examples of suitable propylene-based elastomers may include VERSIFY™ 2000, VERSIFY™ 2200, VERSIFY™ 2300, VERSIFY™ 3200, and VERSIFY™ 3401, which are commercially available from The Dow Chemical Company (Midland, Mich.) or VISTAMAXX™ 6102FL, VISTAMAXX™ 3020FL, which is commercially available from ExxonMobil Chemical Co. (Spring, Tex.).

### Polyethylene Oxide

In addition to the polyolefin elastomer, the film comprises a polyethylene oxide. The term “polyethylene oxide” as used herein includes homo- and copolymers of ethylene oxide. A copolymer may be a random copolymer produced by the polymerization of ethylene oxide mixed with at least one other oxide, such as 1,2-cyclohexene epoxide, 1,2-butene epoxide, allyl glycidyl ether, glycidyl methacrylate, epichlorohydrin, 1,3-butadiene diepoxide, styrene oxide, 4-vinyl-1-cyclohexene 1,2-epoxide, 4-(2-trimethoxysilylethyl)-1,2-epoxycyclohexene and 4-vinyl-1-cyclohexene diepoxide, preferably an alkylene oxide, such as propylene oxide, 1,2-butene epoxide, or isobutylene oxide. Other useful ethylene oxide copolymers are block copolymers produced by the sequential addition of ethylene oxide and at least one other alkylene oxide, in which nearly total consumption of the first monomer takes place prior to the addition of subsequent monomer(s). Alternatively, the ethylene oxide copolymer may comprise in copolymerized form ethylene oxide and another copolymerizable monomer, such as methyl acrylate, ethyl acrylate, a caprolactone, ethylene carbonate, trimethylene carbonate, 1,3-dioxolane, carbon dioxide, carbonyl sulfide, tetrahydrofuran, methyl isocyanate, or methyl isocyanide. Preferred ethylene oxide copolymers are copolymers of ethylene oxide with epichlorohydrin or copolymers of ethylene oxide with cyclohexene oxide. Ethylene oxide copolymers generally comprise at least 50 mol %, preferably at least 70 mol %, more preferably at least 85 mol % ethylene oxide units. The most preferred ethylene oxide polymers are ethylene oxide homopolymers.

The polyethylene oxide has a Brookfield viscosity in 5 wt % aqueous solution at 25° C. of 30 to 115 cP. In some embodiments, the polyethylene oxide has a Brookfield viscosity in 5 wt % aqueous solution at 25° C. of 35 to 115 centiPoise (cP) or 40 to 115 centiPoise (cP), or 40 to 110 cP or 40 to 100 cP or 40 to 95 cP or 45 to 95 cP or 40 to 90 cP. Brookfield viscosity determinations for polyethylene oxide samples were made using a Brookfield DVII+ rotational viscometer (Brookfield Engineering, Middleboro, Mass. USA), 5% aqueous solution, at 25° C. In particular, a sample (30 g) is prepared by stirring in anhydrous iso-propanol (125 mL) to form a slurry, followed by addition of water (475 mL) with stirring for approximately 3 hours at 25.0° C. to form the test solution. After the sample is equilibrated to 25.0° C. in a water bath for 30 minutes, a spindle of specific geometry is immersed into the aqueous sample solution and a well-controlled rotational speed is applied to the spindle for a specified time. The instrument measures the viscous force (torque) that develops on a spring connected to the spindle shaft. Solution viscosity is calculated directly from this measured force. For example POLYOX WSR N10 and POLYOX WSR N80, spindle 1 at 50 rpm was used, with a read time of 0.5 minutes; for the sample POLYOX WSR N750 spindle 2 at 10 rpm was used, with a read time of 1 minute; and for POLYOX WSR N3000 spindle 1 at 2 rpm was used, with a read time of 5 minutes.

Polyethylene oxides having the above described viscosities can have a total average molecular weight of at least 90,000 grams per mole, or 90,000 to 250,000 grams per mole. In some embodiments, the polyethylene oxide can have a molecular weight of at least 100,000 grams per mole, or 100,000 to 250,000 grams per mole. Molecular weight of the polyethylene oxide can be determined using techniques which are generally known such as gel permeation chromatography, light scattering, or rheological measurements. For example, a polyethylene oxide having a Brookfield viscosity in 5 wt % aqueous solution at 25° C. of 30 to 50 cP can have an average molecular weight of 100,000 grams per mole. Such a polyethylene oxide can be obtained as POLYOX WSR N10, available from The Dow Chemical Company.

The polyethylene oxide is present in an amount of 10 to 15 wt %, based on the total weight percent of materials present in the film. Within this range, the polyethylene oxide may be present in an amount of 10 to 14 wt %, or 10 to 13 wt %, or 10 to 12 wt %.

### Hydrophilic Filler

The film further comprises a hydrophilic filler. As used herein, the term “hydrophilic filler” refers to a filler having a hydrophilic surface functionality. Suitable fillers are particulate-type organic or inorganic fillers that have an affinity towards water, by virtue of the hydrophilicity of the functional groups at the surface of the filler. In some embodiments, suitable fillers may have a water contact angle (determined according to ASTM D7334) of 60° or less, preferably 20° or less, more preferably 0° or below the minimum detectable limit for the method. Surface treatments with coatings or coupling agents that reduce the hydrophilicity may impair the affinity of the polyethylene oxide to the filler surface, and hence, are not desired.

Exemplary hydrophilic fillers may include, but are not limited to, aluminum trihydroxide, barium sulfate, calcium carbonate, calcium sulfate, magnesium carbonate, magnesium trihydroxide, diatomaceous earth, dolomite, glass beads, ceramic beads, kaolin, mica, perlite, natural and synthetic silica, wollastonite, whiskers, wood flour, lignin, starch or a combination thereof. In some embodiments, the hydrophilic filler is calcium carbonate (CaCO₃), preferably uncoated calcium carbonate.

The filler particle size distribution is selected such that the largest particle size does not exceed the thickness of the film. The source of enhanced WVTR described herein is associated with enhanced moisture permeation enabled by the presence of the polyethylene oxide and not with microporosity. As such, the filler is not added to create porosity in stretched films, but rather, it is added to stabilize the dispersion of the polyethylene oxide in the film, which is strongly incompatible with the polyolefin elastomer.

The hydrophilic filler is present in an amount of 40 to 60 wt % based on the total weight percent of materials present in the film. Within this range, the filler may be present in an amount of 40 to 55 wt %, 45 to 60 wt %, 45 to 55 wt %, or 45 to 54 wt %.

The film of the present disclosure may also include one or more additives, such as antioxidants (e.g., hindered phenolics such as IRGANOX 1010 or IRGANOX 1076 supplied by Ciba Geigy), phosphites (e.g., IRGAFOS 168, supplied by Ciba Geigy), cling agent (e.g., polyisobutylene (PIB)), phosphonites (e.g., Standostab PEPQ™, supplied by Clariant), pigments, colorants, fillers, TiO2, anti-stat additives, flame retardants, slip agent, antiblock agent, biocides, antimicrobial agents, and the like. Each additive may be included in the film at levels such as 0.01 to 5.0 wt % based on the total weight percent of materials present in the film.

The film having the composition described herein advantageously exhibits breathability and elasticity. Specifically, the film of the present disclosure may have a water vapor transmission rate of at least 1,000 g-mil/m²-day, as determined according to ASTM E398-2780. For example, the film may have a WVTR of 1,000 to 5,000 g-mil/m²-day, or 1500 to 4,000 g-mil/m²-day, or 2,000 to 3,500 g-mil/m²-day.

In some embodiments, the film of the present disclosure may have a permanent set of less than 15% in the machine direction and less than 30% in the cross direction, as determined according to ASTM D5459-95 and after a pre-stretching cycle (in either the machine direction or cross direction) at 200% elongation.

The films may be made using, for example, blown, cast or extrusion coating processes. The films of the present invention will have a total thickness of less than 150, or 125 micrometers, preferably in the range of from 8-100 micrometers, and in another embodiment 12-50 micrometers.

The films of the present disclosure may be used to prepare composite laminates. In some embodiments, the films of the present disclosure may be particularly suited for use in various hygiene articles, for example breathable films used in baby diapers or adult incontinence products, breathable barrier surgical gowns and other hygiene and medical applications.

This disclosure is further illustrated by the following examples, which are non-limiting.

### Examples

Materials used for the following Examples are summarized in Table 1.

TABLE 1

| Component | Description | Supplier |
|---|---|---|
| OBC | Olefin block copolymer having a density of 0.866 g/cm³ and a melt | The Dow Chemical Company |
| | index of 1.0 g/10 min (190° C./2.16 kg) obtained as INFUSE ™ 9107 | |
| PEO-1 | Polyethylene glycol having a kinematic viscosity at 100° C. of 800 | The Dow Chemical Company |
| | centiStokes (according to ASTM D445) obtained as PEG | |
| | CARBOWAX 8000 | |
| PEO-2 | Polyethylene oxide having a Brookfield viscosity in 5 wt % aqueous | The Dow Chemical Company |
| | solution at 25° C. of 3375 cP obtained as POLYOX WSR N3000 | |
| PEO-3 | Polyethylene oxide having a Brookfield viscosity in 5 wt % aqueous | The Dow Chemical Company |
| | solution at 25° C. of 900 cP obtained as POLYOX WSR N750 | |
| PEO-4 | Polyethylene oxide having a Brookfield viscosity in 5 wt % aqueous | The Dow Chemical Company |
| | solution at 25° C. of 40 cP obtained as POLYOX WSR N10 | |
| PEO-5 | Polyethylene oxide having a Brookfield viscosity in 5 wt % aqueous | The Dow Chemical Company |
| | solution at 25° C. of 90 cP obtained as POLYOX WSR N80 | |
| CaCO₃ | Uncoated calcium carbonate obtained as Omyacarb 1-AV calcium | Omya Inc. USA |
| | carbonate, having a median diameter of 1.4 micrometers | |
| Processing | Fluoroelastomer processing aid obtained as Schulman AMF 705 | A. Schulman |
| aid | | |

The compositions of the following Examples were prepared by compounding the components using a BUSS Compounder MDK/E 46 (BUSS S.A. Basel, Switzerland). Prior to use, uncoated calcium carbonate (CaCO₃) was dried at 70° C. overnight. Cooling of the melt for palletization was performed using compressed air. Compounding conditions are summarized in Table 2.

TABLE 2

| T | T | T | T | T | Kneading | ASV | DIE | Cutter |
|---|---|---|---|---|---|---|---|---|
| screw | Z1 | Z2 | ASV | DIE | speed | speed | dimen- | speed |
| (° C.) | (° C.) | (° C.) | (° C.) | (° C.) | (rpm) | (rpm) | sions | (n) |
| 110 | 130 | 135 | 130 | 135 | 110 | 60 | 3 | 5 |

After compounding, the compositions were cooled at 30° C. overnight and then extruded into films. A laboratory Collin cast co-extrusion line equipped with five 30 millimeters extruders was used to produce the samples. All extruders were running the same composition so that conceptually in each case the produced film was equivalent to a monolayer film. The process conditions used to produce all film samples are given in Table 3. Films of 50 micrometer thickness were targeted.

TABLE 3

| Melt | Die | Screw | Take-off | Die |
|---|---|---|---|---|
| temp. | temp. | speed | speed | gap |
| (° C.) | (° C.) | (rpm) | (m/min) | (mm) |
| 203 | 210 | 35 | 5.8 | 0.8 |

The compositions of each Example are summarized in Table 4, where the amount of each component is given in weight percent based on the total weight of the composition.

TABLE 4

| Component | CE1 | CE2 | CE3 | E1 | E2 | E3 | E4 | CE4 |
|---|---|---|---|---|---|---|---|---|
| OBC | 32 | 32 | 32 | 32 | 32 | 41 | 43 | 55 |
| PEO-1 | 12 | | | | | | | |
| PEO-2 | | 12 | | | | | | |
| PEO-3 | | | 12 | | | | | |
| PEO-4 | | | | 12 | | | | |
| PEO-5 | | | | | 12 | 12 | 10 | 8 |
| CaCO₃ | 54 | 54 | 54 | 54 | 54 | 45 | 45 | 35 |
| Processing aid | 2 | 2 | 2 | 2 | 2 | 2 | 2 | 2 |

Films prepared from the compositions in Table 4 were tested tor elastic recovery according to ASTM D5459-95 using an Instron Tensile Tester. Elastic recovery was performed with a pre-stretch cycle of 200%. After release of the pre-stretched sample, a sample of 127 mm length and 15 mm width was cut and then subjected to another hysteresis cycle. This new cycle had two cycles, with each cycle stretched to a target 100% elongation. The permanent set equals the percentage of material that has not recovered to the original length after stretching and is determined using the specimen length at the onset of the second cycle at F=0.1N. The calculation takes into account the length of the specimen after stretching at F=0.1N, lf, and the initial length of the specimen, li, and is as follows:

(lf−li)/li*100=permanent set

Average values of elastic recovery were calculated from a minimum of 5 results and the maximum error accepted was 10%. Measurements were carried out in both machine (MD) and cross directions (CD).

Water vapor transport rate (WVTR) of the films was performed with a Lyssy 80-50000 permeability meter according to ASTM E398-2780. Films were measured at 38° C. and a relative humidity different of 90%. Average values were calculated of 5 measurements of the same sample with a maximum accepted error of 5%. The values were then scaled to 25 micrometer (1 mil) and reported as g-mil/m²-day.

For Comparative Example 1 (CE1), it was not possible to produce a film. The film exhibited strong hole formation during extrusion. Comparative Example 2 (CE2) performed better than CE1, however significant die lines and some holes were observed. It was not possible to produce a homogenous sample that could be used for property testing. Comparative Example 3 (CE3) performed better than CE1 and CE2, but significant die lines were still observed, as well as calcium carbonate agglomerates and an inhomogeneous thickness distribution ranging from 50 to 150 micrometers. It was not possible to produce a homogenous film sample having a thickness of about 50 micrometers from CE3.

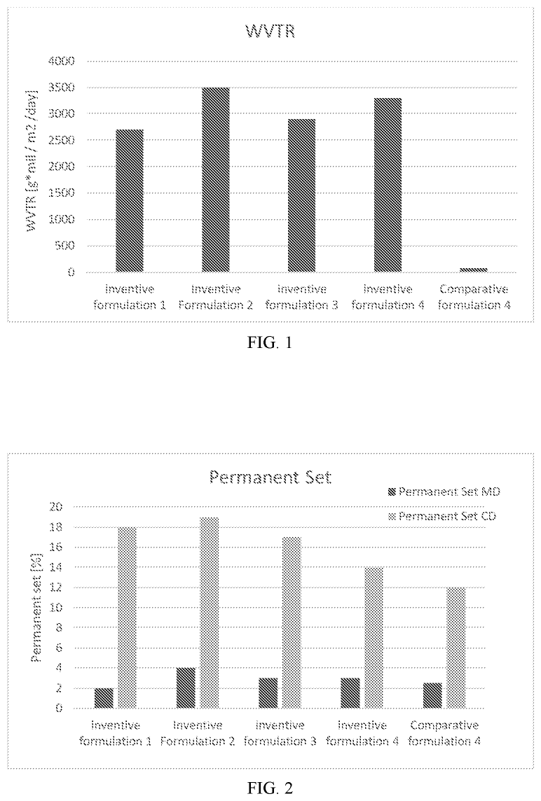

In contrast, when running the compositions of Example 1 and 2 (E1 and E2) it was possible to achieve a significantly better processability and produce film samples having thickness distributions of 48 to 55 micrometers. The measured properties of E1 and E2 indicated that these films possessed good breathability as well as elastic performance. As shown in FIG. 1, the film prepared from the composition of E1 achieved a WVTR of about 2700 g-mil/m²-day and the film prepared from the composition of E2 achieved a WVTR of about 3500 g-mil/m²-day. FIG. 2 shows the elastic performance of E1 and E2 in the machine direction (MD) and the cross direction (CD).

FIGS. 1 and 2 further show the WVTR and elastic properties for E2, E3, E4, and CE4. As may be seen from these Figures, the film from CE4 was not breathable, as evidenced by the low WVTR. These results demonstrate the importance of the amounts of the various components, and that formulations containing more than 35 wt % calcium carbonate and more than 8 wt % polyethylene oxide may achieve the desired properties.

This disclosure further encompasses the following aspects.

Aspect 1: A breathable elastic film comprising: 30 to 45 weight percent of a polyolefin elastomer having a density of 0.860 to 0.890 grams per cubic centimeter; 10 to 15 weight percent of a polyethylene oxide having a Brookfield viscosity in 5 wt % aqueous solution at 25° C. of 30 to 115 cP; and 40 to 60 weight percent of a hydrophilic filler; wherein weight percent is based on the total weight percent of materials present in the film; and wherein the film has a water vapor transmission rate of at least 1,000 g-mil/m²-day, as determined according to ASTM E398-2780.

Aspect 2: The film of aspect 1, wherein the polyolefin elastomer is selected from the group consisting of propylene-based elastomers and ethylene-based elastomers.

Aspect 3: The film of aspect 1 or 2, wherein the polyolefin elastomer is an ethylene/alpha-olefin block copolymer.

Aspect 4: The film of aspect 3, wherein the ethylene/alpha-olefin block copolymer has a melt index of 0.5 to 10.0 grams eluted per 10 minutes, as determined according to ASTM D1238 at 190° C. using a 2.16 kilogram load.

Aspect 5: The film of aspect 1 or 2, wherein the polyolefin elastomer is a propylene/alpha-olefin copolymer.

Aspect 6: The film of aspect 5, wherein the propylene/alpha-olefin copolymer has a melt flow rate of 1.0 to 15.0 grams eluted per 10 minutes, as determined according to ASTM D1238 at 230° C. using a 2.16 kilogram load.

Aspect 7: The film of any one or more of aspects 1 to 6, wherein the polyethylene oxide has a Brookfield viscosity in 5 weight percent aqueous solution at 25° C. of 40 to 115 centiPoise.

Aspect 8: The film of any one or more of aspects 1 to 7, wherein the hydrophilic filler comprises calcium carbonate, mica, kaolin, perlite, diatomaceous earth, dolomite, magnesium carbonate, calcium sulfate, barium sulfate, glass and ceramic beads, natural and synthetic silica, aluminum trihydroxide, magnesium trihydroxide, wollastonite, whiskers, wood flour, lignin, starch or combinations thereof

Aspect 9: The film of any one or more of aspects 1 to 8, wherein the hydrophilic filler comprises uncoated calcium carbonate.

Aspect 10: The film of any one or more of aspects 1 to 9, further comprising one or more additives selected from the group consisting of slip, anti-block, antioxidants, pigments, processing aids, antistats, optical enhancers, phosphites, cling additives, pigments, colorants, flame retardants, biocides, and antimicrobial agents.

Aspect 11: The film of any one or more of aspects 1 to 10, wherein the film exhibits a permanent set of less than 15% in the machine direction and less than 30% in the cross direction, as determined according to ASTM D5459-95 and after a pre-stretch cycle at 200% elongation and wherein the film exhibits a water vapor transmission rate of at least 1,000 g-mil/m²-day, as determined according to ASTM E398-2780.

Aspect 12: The film of aspect 1 to 7, comprising 32 to 43 weight percent of the polyolefin elastomer; 10 to 12 weight percent of the polyethylene oxide; and 45 to 54 weight percent of the hydrophilic filler comprising uncoated calcium carbonate.

Aspect 13: A composite laminate comprising the film of any one or more of aspects 1 to 12.

Aspect 14: A hygiene article comprising the film of any one or more of aspects 1 to 12.

The compositions, methods, and articles may alternatively comprise, consist of, or consist essentially of, any appropriate materials, steps, or components herein disclosed. The compositions, methods, and articles may additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first”, “second”, and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A breathable elastic film comprising:
30 to 45 weight percent of a polyolefin elastomer having a density of 0.860 to 0.890 grams per cubic centimeter;
10 to 15 weight percent of a polyethylene oxide having a Brookfield viscosity in 5 weight percent aqueous solution at 25° C. of 30 to 115 centiPoise; and
40 to 60 weight percent of a hydrophilic filler;
wherein weight percent is based on the total weight percent of materials present in the film; and
wherein the film has a water vapor transmission rate of at least 1,000 g-mil/m²-day, as determined according to ASTM E398-2780.

2. The film of claim 1, wherein the polyolefin elastomer is a propylene-based elastomer, an ethylene-based elastomer, or a combination thereof.

3. The film of claim 1, wherein the polyolefin elastomer is an ethylene/alpha-olefin block copolymer.

4. The film of claim 3, wherein the ethylene/alpha-olefin block copolymer has a melt index of 0.1 to 50.0 grams eluted per 10 minutes, as determined according to ASTM D1238 at 1 90° C. using a 2.16 kilogram load.

5. The film of claim 1, wherein the polyolefin elastomer is a propylene/alpha-olefin copolymer.

6. The film of claim 5, wherein the propylene/alpha-olefin copolymer has a melt flow rate of 1.0 to 15.0 grams eluted per 10 minutes, as determined according to ASTM D1238 at 230° C. using a 2.16 kilogram load.

7. The film of claim 1, wherein the polyethylene oxide has a Brookfield viscosity in 5 weight percent aqueous solution at 25° C. of 40 to 115 centiPoise.

8. The film of claim 1, wherein the hydrophilic filler comprises aluminum trihydroxide, barium sulfate, calcium carbonate, calcium sulfate, magnesium carbonate, magnesium trihydroxide, diatomaceous earth, dolomite, glass beads, ceramic beads, kaolin, mica, perlite, natural and synthetic silica, wollastonite, whiskers, wood flour, lignin, starch or a combination thereof.

9. The film of claim 1, wherein the hydrophilic filler comprises uncoated calcium carbonate.

10. The film of claim 1, further comprising an additive, wherein the additive is an antioxidant, slip agent, anti-block agent, antioxidant, pigment, processing aid, antistat, optical enhancer, phosphite, phosphonite, cling additive, pigment, colorant, filler, Ti02, flame retardant, biocide, antimicrobial agent, or a combination thereof.

11. The film of claim 1, wherein the film exhibits a permanent set of less than 15% in the machine direction and less than 30% in the cross direction, as determined according to ASTM D5459-95 and after a pre-stretch cycle at 200% elongation and the film exhibits a water vapor transmission rate of at least 1,000 g-mil/m²-day, as determined according to ASTM E398-2780.

12. The film of claim 1, comprising
32 to 43 weight percent of the polyolefin
elastomer; 10 to 12 weight percent of the
polyethylene oxide; and
45 to 54 weight percent of the hydrophilic filler comprising uncoated calcium carbonate.

13. A composite laminate comprising the film of claim 1.

14. A hygiene article comprising the film of claim 1.
